(19) United States
(12) Patent Application Publication
(10) Pub. No.: US 2005/0019335 A1
(43) Pub. Date: Jan. 27, 2005

(54) Salmonella vaccine

(76) Inventors: David E Lowery, Kalamazoo, MI (US); Michael J. Kennedy, Galesburg, MI (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/494,919

(22) PCT Filed: Nov. 12, 2001
(86) PCT No.: PCT/IB01/02127

(51) Int. Cl. A61K 39/00; A61K 39/38

(52) U.S. Cl. 424/184.1

(57) ABSTRACT

Attenuated mutant Salmonella bacteria containing inactivated virulence genes are provided for use in vaccines.

### FIELD OF THE INVENTION

The present invention relates generally to genetically engineered salmonellae, which are useful as live vaccines.

### BACKGROUND OF THE INVENTION

Diseases caused by Salmonella bacteria range from a mild, self-limiting diarrhea to serious gastrointestinal and septicemic disease in humans and animals. Salmonella is a gram-negative, rod-shaped, motile bacterium (nonmotile exceptions include S. gallinarum and S. pullorum) that is non-spore forming. Environmental sources of the organism include water, soil, insects, factory surfaces, kitchen surfaces, animal feces, raw meats, raw poultry, and raw seafoods.

Salmonella infection is a widespread occurrence in animals, especially in poultry and swine, and is one of the most economically damaging of the enteric and septicemic diseases that affect food producing animals. Although many serotypes of Salmonella have been isolated from animals, S. choleraesuis and S. typhimurium are the two most frequently isolated serotypes associated with clinical salmonellosis in pigs. In swine, S. typhimurium typically causes an enteric disease, while S. choleraesuis (which is host-adapted to swine) is often the etiologic agent of a fatal septicemic disease with little involvement of the intestinal tract. S. dublin and S. typhimurium are common causes of infection in cattle; of these, S. dublin is host adapted to cattle and is often the etiologic agent of a fatal septicemic disease. Other serotypes such as S. gallinarum and S. pullorum are important etiologic agents of salmonellosis in avian and other species. Although these serotypes primarily infect animals, S. dublin and S. choleraesuis also often cause human disease.

Various Salmonella species have been isolated from the outside of egg shells, including S. enteritidis which may even be present inside the egg yolk. It has been suggested that the presence of the organism in the yolk is due to transmission from the infected layer hen prior to shell deposition. Foods other than eggs have also caused outbreaks of S. enteritidis disease in humans.

S. typhi and S. paratyphi A, B, and C produce typhoid and typhoid-like fever in humans. Although the initial infection with salmonella typically occurs through the gastrointestinal tract, typhoid fever is a systemic disease that spreads throughout the host and can infect multiple organ sites. The fatality rate of typhoid fever can be as high as 10% (compared to less than 1% for most forms of salmonellosis). S. dublin has a 15% mortality rate when the organism causes septicemia in the elderly, and S. eitteritidis has an approximately 3.6% mortality rate in hospital/nursing home outbreaks, with the elderly being particularly affected.

Numerous attempts have been made to protect humans and animals by immunization with a variety of vaccines. Many of the vaccines provide only poor to moderate protection and require large doses to be completely efficacious. Previously used vaccines against sahnonellae and other infectious agents have generally fallen into four categories: (i) specific components from the etiologic agent, including cell fractions or lysates, intact antigens, fragments thereof, or synthetic analogs of naturally occurring antigens or epitopes (often referred to as subunit vaccines); (ii) antiidiotypic antibodies; (iii) the whole killed etiologic agent (often referred to as killed vaccines); or (iv) an avirulent (attenuated) derivative of the etiologic agent used as a live vaccine.

Reports in the literature have shown that attenuated live vaccines are more efficacious than killed vaccines or subunit vaccines for inducing protective immunity. Despite this, high doses of live vaccines are often required for efficacy and few live-attenuated Salmonella vaccines are commercially available. Ideally, an effective attenuated live vaccine retains the ability to infect the host without causing serious disease and is also capable of stimulating humoral (antibody-based) immunity and cell-mediated immunity sufficient to provide resistance to any future infection by virulent bacteria

Several attenuation strategies have been utilized to render Salmonella avirulent [Cardenas et al., Clin Microbial Rev. 5:328-342 (1992); Chatfield et al., Vaccine 7:495-498 (1989); Curtiss, in Woodrow et al., eds., New Generation Vaccines, Marcel Dekker, Inc., New York, p. 161 (1990); Curtiss et al., in Kohler et al., eds., Vaccines: new concepts and developments. Proceedings of the 10th Int'l Convocation of Immunology, Longman Scientific and Technical, Harlow, Essex, UK, pp.261-271 (1987); Curtiss et al., in Blankenship et al., eds., Colonization control of human bacterial enteropathogens in poultry, Academic Press, New York, pp. 169-198 (1991)]. These strategies include the use of temperature sensitive mutants [e.g., Germanier et al., Infect Immun. 4:663-673 (1971)], aromatic and auxotrophic mutants (e.g., -aroA, -asd, -cys, or -thy [Galan et al., Gene 94:29-35 (1990); Hoiseth et al., Nature 291:238-239 (1981); Robertsson et al., Infect Immun. 41:742-750 (1983); Smith et al., Am J Vet Res. 45:59-66 (1984); Smith et al., Am J Vet Res. 45:2231-2235 (1984)]), mutants defective in purine or diaminopimelic acid biosynthesis (e.g., Δpur and Δdap [Clarke et al., Can J Vet Res. 51:32-38 (1987); McFarland et al., Microb Pathog. 3:129-141 (1987); O° Callaghan et al., Infect Immun. 56:419-423 (1988)]), strains altered in the utilization or synthesis of carbohydrates (e.g., ΔgalE [Germanier et al., Infect Immun. 4:663-673 (1971); Hone et al., J Infect Dis. 156:167-174 (1987)]), strains altered in the ability to synthesize lipopolysaccharide (e.g., galE, pmi, rfa) or cured of the virulence plasmid, strains with mutations in one or more virulence genes (e.g., invA) and mutants altered in global gene expression (e.g., -cya -crp, ompR or -phoP [Curtiss (1990), supra; Curtiss et al. (1987), supra; Curtiss et al. (1991)], supra).

In addition, random mutagenesis techniques have been used to identify virulence genes expressed during infection in an animal model. For example, using a variety of approaches, random mutagenesis is carried out on bacteria followed by evaluation of the mutants in animal models or tissue culture systems, such as Signature-Tagged Mutagenesis (STM) [see U.S. Pat. No. 5,876,931].

However, published reports have shown that attempts to attenuate Salmonella by these and other methods have led to varying degrees of success and demonstrated differences in both virulence and immunogenicity [Chatfield et al., Vaccine 7:495-498 (1989); Clarke et al., Can J Vet Res. 51:32-38 (1987); Curtiss (1990), supra; Curtiss et al. (1987), supra; Curtiss et al. (1991), supra]. Prior attempts to use attenuation methodologies to provide safe and efficacious live vaccines have encountered a number of problems.

First, an attenuated strain of Salmonella that exhibits partial or complete reduction in virulence may not retain the ability to induce a protective immune response when given as a vaccine. For instance, ΔaroA mutants and galE mutants of S. typhimurium lacking UDP-galactose epimerase activity were found to be immunogenic in mice [Germanier et al., Infect Immun. 4:663-673 (1971), Hohmann et al., Infect Imun. 25:27-33 (1979); Hoiseth et al., Nature, 291:238-239 (1981); Hone et al., J. Infect Dis. 156:167-174 (1987)] whereas Δasd, Δthy, and Δpur mutants of S. typhimurium were not [Curtiss et al. (1987), supra, Nnalue et al., Infect Immun. 55:955-962 (1987)]. All of these strains, nonetheless, were attenuated for mice when given orally or parenterally in doses sufficient to kill mice with the wild-type parent strain. Similarly, ΔaroA, Δasd, Δthy, and Δpur mutants of S. choleraesuis were avirulent in mice, but only ΔaroA mutants were sufficiently avirulent and none were effective as live vaccines [Nnalue et al., Infect Immun. 54:635-640 (1986); Nnalue et al., Infect Immun. 55:955-962 (1987)].

Second, attenuated strains of S. dublin carrying mutations in phoP, phoP crp, [crp-cdt] cya, crp cya were found to be immunogenic in mice but not cattle [Kennedy et al., Abstracts of the 97th General Meeting of the American Society for Microbiology. B-287:78 (1997)]. Likewise, another strain of S. dublin, SL5631, with a deletion affecting gene aroA was highly protective against lethal challenge to a heterologous challenge strain in mice [Lindberg et al., Infect Immun. 61:1211-1221 (1993)] but not cattle [Smith et al., Am J Vet Res. 54:1249-1255 (1993)].

Third, genetically engineered Salmonella strains that contain a mutation in only a single gene may spontaneously mutate and “revert” to the virulent state. The introduction of mutations in two or more genes tends to provide a high level of safety against restoration of pathogenicity by recombination [Tacket et al., Infect Immun. 60:536-541 (1992)]. However, the use of double or multiple gene disruptions is unpredictable in its effect on virulence and immunogenicity; the introduction of multiple mutations may overattenuate a bacteria for a particular host [Linde et al., Vaccine 8:278-282 (1990); Zhang et al., Microb. Pathog., 26(3):121-130 (1999)].

The present invention relates to a Salmonella cell the virulence of which is attenuated by a disruption or deletion of all or a portion of the waaK (formerly rfaK) gene. Homologs to the Salmonella waaK gene have been discovered in several gram-negative bacteria, including the Neisseria meningitidis rfaK gene (Rahman et al, Glycoprotein 11:703-709. 2001), where it was shown to encode for a N-acetylglucosamine transferase involved in the proper assembly of the N. meningitidis lipooligosaccharide (LOS) protein. Similar to the rfaK gene of N. meningitidis, the Salmonella waaK gene appears to play a role in proper assembly of the gram-negative bacteria lipopolysaccharide (LPS) protein, which is structurally different than the LOS proteins.

To date, most Salmonella vaccines typically give strong serotype-specific protection but offer limited or no cross-protection against different serogroups of Salmonella. The present invention, which demonstrates a disruption in a gene common to many serotypes of Salmonella and necessary for bacterial virulence, may offer a broad cross-protective vaccine across salmonella serogroups and possibly other gram-negative enteric bacterial pathogens. Vaccines composed of bacteria outlined in the present patent application may give other uses such as salmonella as a vector for antigen or DNA delivery.

A need continues to exist for more safe and efficacious live attenuated Salmonella vaccines that ideally do not need to be administered at very large doses. The invention also features vaccines comprising such attenuated bacteria vaccine for the vaccination of poultry and mammals against a variety of gram negative pathogens belonging to Enterobacteriaceae, and in particular the genus Salmonella.

### BRIEF SUMMARY OF THE INVENTION

The present invention relates to safe and efficacious vaccines employing one or more strains of attenuated mutant gram-negative bacteria in which one or more genes homologous to genes of Salmonella waaK (formerly rfaK) have been inactivated, preferably by deletion of about 5% to about 100% of the gene, most preferably by deletion of about 50% or more of the gene. Specifically contemplated are vaccines comprising one or more species of attenuated mutant Salmonella bacteria in which one or more genes, and preferably two or more genes, homologous to waaK have been inactivated. Also contemplated by the invention are mutations generated by an insertion into the virulence gene. In a preferred embodiment, particularly waaK genes have been inactivated in the mutant bacteria. Preferably, the vaccine composition of the invention comprises a vaccine wherein the inactivated gene is selected from the group consisting of:

ctcaatcact tatcaaacca gtttttcatt tgttcctcga aacgctgcgc tacattttcc
caactgtatt ttgaaaacac cagggatttt gctttttcgg caatctggtg gcgttcctta
tcagcaagcg cacggttaat atcattaatt atactgtcgc tcgacatagg ttctgcgagg
tgatagcccg ttatgccatc taacacaaat tcgctaatcc cccctttttt gctggcaaga
accgcttttc ctgctgccat cgcttctaca gccaccatgc aaaatgcttc ttcaacctga
getggcacaa taaccagatc ggctatatga tagaagttat gcatctggtc aggagattgc
cccccagcca taatacaatc cgttccaatc tcttttgcgg cgtccagtac tttcttttga
tactctgctt tttcaccctt gcggcttgca taagggtcgc caacaacgac aagtttaata
ttacttctta aggtacgtaa ttgtttgaac gcctgcaaaa gcaacaggat gcctttatca
ggcgaaattc tcccggcata caagagaacg gtggcatctt ccgcaatatt taattgctga
cgaagattat cttgtgggtt tcttttataa gtctcagcac aaaaaccatt aggcacaata
ctaacagcag cggcgggcaa tctttcttca taaaacgctt taagaaactg actgggcacg
ataatttttg catcattatc aggaagttct ggttcaaatg cattatgcat gtgcataacc
agttttgcat tcggattgcg ctctctgatc tgccgataca gtttcatact attatgaata
acaatgacgc tatcttcctg ggtagtcact ttatctctaa tattaaggat gcgctgggaa
tagggtagtg ggtcgagacg agtccatttc tgaaaaagac gcttataaac tttactaaac
ccgatgtaat gaatatcaca gttatcgttt attttattat attcaggata gccagcattc
tttatacaag caatagcatt cggtattgat agtcgttttg caacctggta aatccaggtt
tctaccgcag ccgcaccacg aggaggaatt gaaaatatag gagtaacagt aaatatgatt
tttttaatca taatagctat aatcc

b) a full length nucleotide sequence that hybridizes to the non-coding complement of the SEQ. ID NO. 1 and; c) a fall length Salmonella nucleotide sequence that has 95% sequence identity to SEQ. ID NO. 1.

The invention is based on results of extensive safety and efficacy testing of these vaccines, including vaccines containing more than one serotype of Salmonella, in animal species other than rodents, including cattle and pigs.

According to one aspect of the present invention, vaccine compositions are provided that comprise an immunologically protective amount, of a first attenuated mutant Salmonella bacterium in which one or more waaK genes are inactivated. In one embodiment, the genes are selected from the group consisting of waaK. Suitable amounts will vary but may include about 10⁹ bacteria or less. In these mutant bacteria, the inactivated gene(s) is/are preferably inactivated by deletion of a portion of the coding region of the gene. Alternatively, inactivation is effected by insertional mutation. Any species of Salmonella bacteria, particularly S. enterica subspecies and subtypes, may be mutated according to the invention, including Salmonella from serogroups A, B, C₁, C₂, D₁ and E₁. All of the Salmonella serovars belong to two species: S. bongori and S. enterica. The six subspecies of S. enterica are: S. enterica subsp. enterica (I or 1), S. enterica subsp. salamae (II or 2), S. enterica subsp. arizonae (ma or 3a), S. enterica subsp. diarizonae (IIIb or 3b), S. enterica subsp. houtenae (IV or 4), S. enterica subsp. indica (VI or 6). Exemplary subspecies include: S. Choleraesuis, S. Typhimurium, S. Typhi, S. Paratyphi, S. Dublin, S. Enteritidis, S. Gallinarum, S. Pullorum, Salmonella Anatum, Salmonella Hadar, Salmonella Hamburg, Salmonella Kentucky, Salmonella Miami, Salmonella Montevideo, Salmonella Ohio, Salmonella Sendai, Salmonella Typhisuis.

Two or more virulence genes may be inactivated in the mutant Salmonella bacteria, of which at least one gene is a waaK gene.

The vaccine composition may further comprise a second attenuated mutant Salmonella bacterium in which one or more virulence genes have been inactivated. Preferably, the first and second mutant Salmonella bacteria are of different serotypes. For cattle, vaccines comprising both S. dublin and S. typhimurium are preferred.

The invention also provides methods of immunizing, i.e., conferring protective immunity on, an animal by administering the vaccine compositions of the invention, wherein the immunologically protective amount of attenuated bacterium provides an improvement in mortality, symptomatic diarrhea, physical condition or milk production. The invention further provides methods of reducing transmission of infection by administering vaccines of the invention in amounts effective to reduce amount or duration of bacterial shedding during infection. Animals that are suitable recipients of such vaccines include but are not limited to cattle, sheep, horses, pigs, poultry and other birds, cats, dogs, and humans. Methods of the invention utilize any of the vaccine compositions of the invention, and preferably, the vaccine comprises an effective amount of an attenuated, non-reverting mutant Salmonella bacterium in which one or more waaK genes have been inactivated, either by deleting a portion of the gene(s), or, alternatively, by insertional mutation.

According to another aspect of the invention, the attenuated mutant Salmonella bacterium may further comprise a polynucleotide encoding a non-Salmonella polypeptide. Administration of the mutant bacteria or a vaccine composition comprising the mutant bacteria thus provides a method of delivering an immunogenic polypeptide antigen to an animal.

Numerous additional aspects and advantages of the invention will become apparent to those skilled in the art upon consideration of the following detailed description of the invention which describes presently preferred embodiments thereof.

### DETAILED DESCRIPTION OF THE INVENTION

The present invention provides vaccines, or immunogenic compositions, comprising one or more species of attenuated mutant Salmonella bacteria in which one or more virulence genes, preferably the waaK genes have been deleted. An advantage of the vaccines of the present invention is that the live -attenuated mutant bacteria can be administered as vaccines at reasonable doses, via a variety of different routes, and still induce protective immunity in the vaccinated animals. Another advantage is that mutant bacteria containing inactivations in two different genes are non-reverting, or at least are much less likely to revert to a virulent state.

Risk of reversion can be assessed by passaging the bacteria multiple times (e.g., 5 passages) and administering the resulting bacteria to animals. Non-reverting mutants will continue to be attenuated.

The examples herein demonstrate that inactivation or deletion of the waaK gene results in safe, efficacious vaccines as shown by observable reductions in adverse signs and symptoms associated with infection by wild type bacteria. The exemplary vaccines of the present invention have been shown to confer superior protective immunity compared to other vaccines containing live attenuated bacteria, e.g., Salmo Shield®TD (Grand Laboratories, Inc.) and mutant Salmonella bacteria containing Δcya Δcrp mutations (χ³⁷⁸¹).

The nucleotide sequence of waaK from S. thyphimurium is set forth in SEQ ID NO: 1. As used herein, “waaK” includes SEQ ID NO: 1 and other Salmonella species equivalents thereof, e.g., full length Salmonella nucleotide sequences that hybridize to the non coding complement of SEQ ID NO: 1 under stringent conditions (e.g., as described in FIG. 4 of Shea et al., Proc. Nat'l. Acad. Sci. USA, 93:2593-2597 (1996), incorporated herein by reference), and full length Salmonella nucleotide sequences that have 90% sequence identity to SEQ ID NO: 1 or 2. Salmonella species equivalents can be easily identified by those of ordinary skill in the art and also include nucleotide sequences with, e.g. 90%, 95%, 98% and 99% identity to SEQ ID NO: 1

The invention also contemplates that equivalent genes (e.g., greater than 80% homology) in other gram negative bacteria can be similarly inactivated to provide efficacious vaccines.

As used herein, an “inactivated” gene means that the gene has been mutated by insertion, deletion or substitution of nucleotide sequence such that the mutation inhibits or abolishes expression and/or biological activity of the encoded gene product. The mutation may act through affecting transcription or translation of the gene or its mRNA, or the mutation may affect the polypeptide gene product itself in such a way as to render it inactive.

In preferred embodiments, inactivation is carried by deletion of a portion of the coding region of the gene, because a deletion mutation reduces the risk that the mutant will revert to a virulent state. For example, some, most (e.g., half or more) or virtually all of the coding region may be deleted (e.g., about 5% to about 100% of the gene, but preferably about 20% or more of the gene, and most preferably about 50% or more of the gene may be deleted). Alternatively, the mutation may be an insertion or deletion of even a single nucleotide that causes a frame shift in the open reading frame, which in turn may cause premature termination of the encoded polypeptide or expression of an completely inactive polypeptide. Mutations can also be generated through insertion of foreign gene sequences, e.g., the insertion of a gene encoding antibiotic resistance.

Deletion mutants can be constructed using any of a number of techniques well known and routinely practiced in the art. In one example, a strategy using counterselectable markers can be employed which has commonly been utilized to delete genes in many bacteria. For a review, see, for example, Reyrat, et al., Infection and Immunity 66:4011-4017 (1998), incorporated herein by reference. In this technique, a double selection strategy is often employed wherein a plasmid is constructed encoding both a selectable and counterselectable marker, with flanking DNA sequences, derived from both sides of the desired deletion. The selectable marker is used to select for bacteria in which the plasmid has integrated into the genome in the appropriate location and manner. The counterselecteable marker is used to select for the very small percentage of bacteria that have spontaneously eliminated the integrated plasmid. A fraction of these bacteria will then contain only the desired deletion with no other foreign DNA present. The key to the use of this technique is the availability of a suitable counterselectable marker.

In another technique, the cre-lox system is used for site specific recombination of DNA. The system consists of 34 base pair lox sequences that are recognized by the bacterial cre recombinase gene. If the lox sites are present in the DNA in an appropriate orientation, DNA flanked by the lox sites will be excised by the cre recombinase, resulting in the deletion of all sequences except for one remaining copy of the lox sequence. Using standard recombination techniques, it is possible to delete the targeted gene of interest in the Salmonella genome and to replace it with a selectable marker (e.g., a gene coding for kanamycin resistance) that is flanked by the lox sites. Transient expression (by electroporation of a suicide plasmid containing the cre gene under control of a promoter that functions in Salmonella of the cre recombinase should result in efficient elimination of the lox flanked marker. This process would result in a mutant containing the desired deletion mutation and one copy of the lox sequences.

In another approach, it is possible to directly replace a desired deleted sequence in the Salmonella genome with a marker gene, such as green fluorescent protein (GFP), β-galactosidase, or luciferase. In this technique, DNA segments flanked marker. This process would result in a mutant containing the desired deletion vector for Salmonella. An expression cassette, containing a promoter active in Salmonella and the appropriate marker gene, is cloned between the flanking sequences. The plasmid is introduced into wild-type Salmonella. Bacteria that incorporate and express the marker gene (probably at a very low frequency) are isolated and examined for the appropriate recombination event (i.e., replacement of the wild type gene with the marker gene).

In order for a modified strain to be effective in a vaccine formulation, the attenuation must be significant enough to prevent the pathogen from evoking severe clinical symptoms, but also insignificant enough to allow limited replication and growth of the bacteria in the recipient. The recipient is a subject needing protection from a disease caused by a virulent form of Salmonella or other pathogenic microorganisms. The subject to be immunized maybe a humnan or other mammal or animal, for example, farm animals including cows, sheep, pigs, horses, goats and poultry (e.g., chickens, turkeys, ducks and geese) and companion animals such as dogs and cats; exotic and/or zoo animals. Immunization of both rodents and non-rodent animals is contemplated.

An “immunologically protective amount of the attenuated mutant bacteria is an amount effective to induce an immunogenic response in the recipient that is adequate to prevent or ameliorate signs or symptoms of disease, including adverse health effects or complications thereof, caused by infection with wild type Salmonella bacteria. Either humoral immunity or cell-mediated immunity or both may be induced. The immunogenic response of an animal to a vaccine composition may be evaluated., e.g., indirectly through measurement of antibody titers, lymphocyte proliferation assays, or directly through monitoring signs and symptoms after challenge with wild type strain.

The protective immunity conferred by a vaccine can be evaluated by measuring, e.g., reduction in clinical signs such as mortality, morbidity, temperature number and % of days of diarrhea, milk production or yield, average daily weight gain [ADG=([Inoculation weight−Vaccination weight)/(Inoculation date−Vaccination date)], physical condition and overall health and performance of the subject.

When a combination of two or more different serotypes of bacteria are administered, it is highly desirable that there be little or no interference among the serotypes such that the host is not prevented from developing a protective immune response to one of the two or more serotypes administered. Interference can arise, e.g., if one strain predominates in the host to the point that it prevents or limits the host from developing a protective immune response to the other strain. Alternatively, one strain may directly inhibit the other strain.

In addition to immunizing the recipient, the vaccines of the invention may also promote growth of the recipient and/or boost the recipient's immunity and/or improve the recipient's overall health status. Components of the vaccines of the invention, or microbial products, may act as immunomodulators that may inhibit or enhance aspects of the immune system. For example, the vaccines of the invention may signal pathways that would recruit cytokines that would have an overall positive benefit to the host.

The vaccines of the present invention also provide veterinary and human community health benefit by reducing the shedding of virulent bacteria by infected animals. Either bacterial load being shed (the amount of bacteria, e.g., CFU/ml feces) or the duration of shedding (e.g., number of % of days shedding is observed) may be reduced, or both. Preferably, shedding load is reduced by about 10% or more compared to unvaccinated animals preferably by 20% or more, and/or shedding duration is reduced by at least 1 day, or more preferably 2 or 3 days, or by 10% or more or 20% or more.

While it is possible for an attenuated bacteria of the invention to be administered alone, one or more of such mutant bacteria are preferably administered in conjunction with suitable pharmaceutically acceptable excipient(s), diluent(s), adjuvant(s) or carrier(s). The carrier(s) must be “acceptable” in the sense of being compatible with the attenuated mutant bacteria of the invention and not deleterious to the subject to be immunized. Typically, the carriers will be water or saline which will be sterile and pyrogen free.

Any adjuvant known in the art may be used in the vaccine composition, including oil-based adjuvants such as Freund's Complete Adjuvant and Freund's Incomplete Adjuvant, mycolate-based adjuvants (e.g., trehalose dimycolate), bacterial lipopolysaccharide (LPS), peptidoglycans (i.e., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), Biostim™(e.g., 01K2), the “Iscoms” of EP 109 942, EP 180 564 and EP 231 039, aluminum hydroxide, saponin, DEAE-dextran, neutral oils (such as miglyol), vegetable oils (such as arachis oil), liposomes, Pluronic® polyols, the Ribi adjuvant system (see, for example GB-A-2 189 141), or interleukins, particularly those that stimulate cell mediated immunity. An alternative adjuvant consisting of extracts of Amycolata, a bacterial genus in the order Actinomycetales, has been described in U.S. Pat. No. 4,877,612. Additionally, proprietary adjuvant mixtures are commercially available. The adjuvant used will depend, in part, on the recipient organism. The amount of adjuvant to administer will depend on the type and size of animal. Optimal dosages may be readily determined by routine methods.

The vaccine compositions optionally may include vaccine-compatible pharmaceutically acceptable (ie., sterile and non-toxic) liquid, semisolid, or solid diluents that serve as pharmaceutical vehicles, excipients, or media. Any diluent known in the art may be used. Exemplary diluents include, but are not limited to, polyoxyethylene sorbitan monolaurate, magnesium stearate, methyl- and propylhydroxybenzoate, talc, alginates, starches, lactose, sucrose, dextrose, sorbitol, mannitol, gum acacia, calcium phosphate, mineral oil, cocoa butter, and oil of theobroma.

The vaccine compositions can be packaged in forms convenient for delivery. The compositions can be enclosed within a capsule, caplet, sachet, cachet, gelatin, paper, or other container. These delivery forms are preferred when compatible with entry of the immunogenic composition into the recipient organism and, particularly, when the immunogenic composition is being delivered in unit dose form. The dosage units can be packaged, e.g., in tablets, capsules, suppositories or cachets.

The vaccine compositions may be introduced into the subject to be immunized by any conventional method including, e.g., by intravenous, intradermal, intramuscular, intramammary, intraperitoneal, or subcutaneous injection; by oral, transdermal, sublingual, intranasal, anal, or vaginal, delivery. The treatment may consist of a single dose or a plurality of doses over a period of time.

Depending on the route of administration, suitable amounts of the mutant bacteria to be administered include ˜10⁹ bacteria or less, provided that an adequate immunogenic response is induced by the vaccinee. Doses of ˜10¹⁰ or less or ˜10¹¹ or less may be required to achieve the desired response. Doses significantly higher than ˜10¹¹ may not be commercially desirable.

Another aspect of the invention involves the construction of attenuated mutant bacteria that additionally comprise a polynucleotide sequence encoding a heterologous polypeptide. For example, for Salmonella, a “heterologous” polypeptide would be a non-Salmonella polypeptide not normally expressed by Salmonella bacteria. Such attenuated mutant bacteria can be used in methods for delivering the heterologous polypeptide or DNA. For example, Salmonella could be engineered to lyse upon entry into the cytoplasm of a eukaryotic host cell without causing significant damage, thereby becoming a vector for the introduction of plasmid DNA into the cell. Suitable heterologous polypeptides include immunogenic antigens from other infectious agents (including gram-negative bacteria, gram-positive bacteria and viruses) that induce a protective immune response in the recipients, and expression of the polypeptide antigen by the mutant bacteria in the vaccine causes the recipient to be immunized against the antigen. Other heterologous polypeptides that can be introduced using the mutant Salmonella include immunomodulatory molecules e.g., cytokines or “performance” proteins such as growth hormone, GRH, and GDF-8.

### EXAMPLE 1

### Construction of Salmonella Mutants Containing Deletions of waaK

A. Construction of pCVD442::ΔGene Plasmids.

For each of the S. typhizurium waaK genes, positive selection suicide vectors based on the plasmid pCVD442 [Donnenberg and Kaper, Infect Immun 59:4310-17 (1991)] were constructed that contained a portion of the 5′ and 3′ chromosomal regions flanking each gene but with substantial internal deletions (typically >95%) within the gene itself. Gene splicing by overlap extension (“gene SOEing” [Horton et al., Biotechniques 8:528-535 (1990)]) was used to generate DNA fragments which were complementary to the gene to be deleted, but which lacked the majority of the internal nucleotide sequence. The plasmids containing these internally deleted genes were designated pCVD442::ΔssaJ, and pCVD442::ΔrfaK, respectively. These vectors were then used to generate S. typhimurium and S. dublin deletion mutants by allelic exchange. These plasmids are also described in WO 01/70247A2, incorporated herein by reference. Plasmids containing the S. dublin deleted genes were used to produce the deletions in S. dublin, and plasmids containing the S. typhirinrium sequences were used to produce the deletions in S. thyphimurium (see Example 1B below).

In brief, two sets of PCR primers were designed to synthesize approximately 600 bp fragments that are complementary to the DNA flanking the 5′ and 3′ sides of the desired gene. Primers A and D (Table 1) contain chromosomal sequence upstream and downstream, respectively, of the desired gene and each also contains the nucleotide sequence for a desired restriction endonuclease site. Primer B spans the upstream junction between the sequences immediately flanking the 5′ side of the gene and the gene itself and includes some a portion of the 5′ end of the gene (in some cases, only the stop codon). Similarly, primer C spans the downstream junction between the sequences immediately flanking the 3′ side of the gene and the gene itself, and includes a portion of the 3′ end of the gene (in some cases, only the start codon). PCR reactions with S. thyphimurium or S. dublin genomic DNA and either primers A and B or primers C and D were performed, yielding PCR products (designated fragments AB and CD, respectively) of approximately 600 bp with sequences corresponding to the upstream or downstream flanking regions of the desired gene, respectively. Each AB or CD fragment also contained the desired restriction site (Sal I for rfaK). A second PCR reaction using fragments AB and CD with primers A and D was then performed, yielding a PCR product designated fragment AD. Fragment AD is complementary to the nucleotide sequence surrounding the targeted gene, but contains essentially a complete deletion of the targeted sequences (>95% deletion) for waaK, and a deletion of the C-terminal half (˜50% deletion) for ssaJ. The resulting PCR product for each of the S. dublin or S. typhiniurium waaK gene was then cloned through various vectors and host strains and finally inserted into the multiple cloning site of vector pCVD442 in host strain SM10λpir.

TABLE 1
Primers used in construction of modified S. typhimurium genes*.

| | Primer | | | |
|---|---|---|---|---|
| | Number | Sequence ID | | Restriction |
| Gene | (Letter) | NOs: | Primer Sequence | Site |
| rfaK | 882 (A) | 2 | 5′-GCCAAGTCGACATAGTAGGTGTTCTGTGGGCAATA-3′ | SalI |
| | 883 (B) | 3 | 5′-TTCTGGATTATAGCTATTATGATTGTTTGATAAGTGATTGAGTCCTGA-3′ | — |
| | 884 (C) | 4 | 5′-TCAGGACTCAATCACTTATCAAACAATCATAATAGCTATAATCCAGAA-3′ | — |
| | 885 (D) | 5 | 5′-GCCAAGTCGACGTGTACGAACAGGCTTCAGTGGAT-3′ | SalI |

*PCR primers used in generating the left (5′) and right (3′) flanking regions of the rfaK gene. Primers A/B and C/D are the 5′ and 3′ primer sets, respectively, for that gene. Primers A and D are the primers that are the furthest upstream and downstream from that gene and were designed to incorporate the restriction sites indicated into the PCR product.

The S. dublin and S. thyphimurium genes are similar enough that the same primers could be used for both serotypes.

B. Construction of Deletion Mutants of S. thyphimurium and S. dublin

The pCVD442::Δgene plasmids constructed in Example 1A above were used to produce deletion mutants by homologous recombination with the appropriate Salmonella strain i.e., a plasmid containing the S. dublin deleted gene was used to produce the deletion in S. dublin, and a plasmid containing the S. typhimirium sequences was used to produce the deletion in S. typhimurium. The plasmid pCVD442 is a positive selection suicide vector. It contains the origin of replication for R6K plasmids (ori), the mobilization gene for RP4 plasmids (nmob), the gene for ampicillin resistance (bla), the sacB gene from B. subtilis, which encodes the gene for levan sucrase and a multiple cloning site.

The plasmid pCVD442 can be maintained extrachromosomally only in bacterial strains producing the π protein, the pir gene product (e.g. E. coli SM10λpir or DH5απpir). Introduction of a pCVD442 based vector into a nonpermissive host strain (S. thyphimurium or S. dublin), by conjugation and selection on Ap (ampicillin) and Nal (nalidixic acid) containing medium, allows the isolation of Ap^{R} merodiploid isolates in which the plasmid has integrated into the genome of the target strain by homologous recombination with the wild type gene.

In brief, E. coli strain SM10λpir (thi thr leu tonA lacY supE recA::RP4-2-Tc::Mu km)[(Donnenberg and Kaper, Infect Immun 59:4310-17 (1991)] carrying the pCVD442 plasmids with the S. typhimurium or S. dublin ΔssaT, ΔssaJ, ΔssaC, ΔrfaK or ΔglnA genes (designated SM10λpir/pCVD442::Δgene) were mated with Nal^{R} S. typhimurium MK315N or S. dublin B94-058N, and recombinants were selected on Ap and Na1. Both MK315N and B94-058N are spontaneous Na1^{R} strains prepared by plating the respective parent strains on LB agar containing 50 μg/ml Na1 (clinical isolates from a bovine and a human subject, respectively). The Ap^{R} Na1^{R} recombinants recovered must have the plasmid integrated into the chromosome because the plasmid cannot be maintained extrachromosomally. This results in the formation of a merodiploid strain that contains the pCVD442::Δgene plasmid integrated into that gene locus on the chromosome.

The Ap^{R} Na1^{R} S. thyphimurium M 315N::pCVD442::Δgene and S. dublin B94-058N::pCVD442::Δgene recombinants were then grown under non-selective conditions followed by growth on LA (−sucrose) and TYES (+sucrose) agar. In the absence of selection pressure a spontaneous recombination event can occur in which the pCVD442 plasmid and either the wild-type gene or the deleted gene are excised from the chromosome. Cells retaining the pCVD442 plasmid were counterselected on TYES agar by the toxic products produced from the breakdown of sucrose by levan sucrase, encoded by the sacB gene. Consequently, the number of colonies on TYES agar is significantly reduced relative to the number on LA. After confirming the Ap^{S} phenotype of the isolated colonies on the TYES agar, the recombinants were analyzed by PCR to determine whether the wild-type gene or the deleted gene had been retained.

In initial experiments, the donor and recipient were mated for 5 hrs. on LB agar and then selected on LB agar containing Na1 (20 or 100 μg/ml) and Ap (20 or 100 μg/ml). While heavy growth appeared on the initial selection plate few, if any, of the isolated colonies could be confirmed as Aap^{R} Na1^{R}. The inability to isolate recombinant growth was likely due to the growth of the recipient as a result of the degradation of ampicillin by the release of β-lactamase from the donor cells. To overcome this problem, mating and selection conditions were designed that favored the recombinants and selected against the donor and recipient strains. Specifically, recipient and donor strains were mated overnight on LB agar or modified M9 agar (Difco Laboratory, Detriot, Mich.], followed by enrichment of recombinants by growth in selective (Na1 and Ap (75 μg/ml)) LB broth (Difco Laboratory, Detriot, Mich.), and isolation on selective (Na1 and Ap (75 μg/ml)) agar medium. Mating on modified M9 agar allowed conjugation to occur, but limited replication, which reduced the number of donor and recipient cells introduced to the selection broth. Growth to early logarithmic phase in selection broth favored the replication of the recombinants but not the donor and recipient strains. Subsequent selection on LB agar Na1 Ap (75 μg/ml each) further favored the recombinants over the donor and recipient, which was confirmed when almost all isolated colonies were Aap^{R} Na1^{R}. This procedure yielded merodiploid S. thyphimurium or S. dublin recombinants carrying the appropriate plasmid pCVD442::Δgene inserted into the genome.

Meridiploid isolates were then grown under non-selective conditions to late logarithmic phase and inoculated to LB agar and TYES agar. During non-selective growth a spontaneous recombination event can occur between the duplicated sequences in the merodiploid state, leaving a copy of either the wild type or deleted gene in the chromosome. Growth on sucrose (TYES) selects against those cells which have not undergone the second recombination event because the products of levan sucrase, encoded by the sacB gene on the pCVD442 plasmid, are toxic to gram-negative cells. Consequently, the number of colonies on TYES agar is much lower than on LA. In our hands, it was critical to incubate the TYES plates at room temperature for the selection to be successful. Incubation at higher temperatures (30° or 37° C.) did not reduce the number of colonies on TYES relative to LA indicating that selection for pCVD442-negative cells did not occur.

TYES-grown colonies were streaked for single colony and the Na1^{R} Ap^{S} phenotype confirmed. PCR analysis of the genomic DNA of the colonies using the appropriate Primers A and D described above for each gene was then performed to determine whether the deleted or wild type gene had been retained in the chromosome. For waaK, a PCR product of 1300 bp (vs. 2400 bp for wild type gene) indicated that the gene had been deleted.

C. Construction of S. chioleraesuis Mutants

S. choleraesuis mutants were constructed using the STM process generally described in U.S. Pat. No. 5,876,931, incorporated herein by reference. Briefly, each insertional mutation produced carries a different DNA signature tag, which allows mutants to be differentiated from each other. The tags comprise 40-bp variable central regions flanked by invariant “arms” of 20-bp which allow the central portions to be co-amplified by PCR. Tagged mutant strains are assembled in microtiter dishes, then combined to form the “inoculum pool” for infection studies. At an appropriate time after inoculation, bacteria are isolated from the animal and pooled to form the “recovered pool.” The tags in the recovered pool and the tags in the inoculum pool are separately amplified, labeled, and then used to probe filters arrayed with the different tags representing the mutants in the inoculum. Mutants with attenuated virulence are those with tags that give hybridization signals when probed with tags from the inoculum pool but not when probed with tags from the recovered pool. STM allows a large number of insertional mutant strains to be screened simultaneously in a single animal for loss of virulence. Using this method, insertional mutants of S. choleraesuis containing a mini-tn5 transposon interrupting the particular gene were generated. Portions of the gene surrounding each transposon were sequenced to identify the insertion site by alignment of the sequence with the corresponding sequence of known S. thyphimurium genes.

### EXAMPLE2

### Determination of S. Clholeraesuis of Mutant Attenuation and LD₅₀ in Mice

To determine the degree of attenuation, groups of six mice (BALB/c) were infected with each individual mutant and a range of doses, 8×10² to 8×10⁶ for oral administration and a range 10-fold less for intra peritoneal (IP) administration. The attenuated mutants shoe different degrees of attenuation based on LD₅₀ values when compared to the wild type strain. Mutants D1 and H5 which contain the Tn5 transposon insertion demonstrate attenuation three to four orders of magnitude less than the wild type strain. Mutant D1 has an LD₅₀ of 5.2×10⁷ when given orally and 3.9×10³ when administered IP compared to the wild type LD₅₀ values of 1.1×10³ orally and 2.6×10² when given IP. The H5 mutant gives and LD₅₀ of 7.9×10⁴ orally and 3.0×10⁴ as an IP vaccine. These results from BALB/c mice demonstrate that the D1 and H5 waaK mutants show a significantly reduced LD50 and greater than 50-fold measured attenuation of the bacteria when compared to wild type S. choleraesuis.

### EXAMPLE3

Safety and Efficacy of waaK Deletion Mutants

A. Efficacy of a S. chioleraesuis wwak Mutant as Vaccines in Swine

(Trial No. 704-7923-I-MJK-96-012)

The safety and efficacy of a live attenuated S. choleraesuis waaK mutants as a vaccine was determined in swine (8 pigs per group, 18-24 days of age at vaccination). Baseline temperatures and were recorded on Days 1-4. Baseline values for body temperatures, fecal consistency, and physical condition for each animal were collected during the four days immediately prior to vaccination, and were compared to post-vaccination values to assess the safety of each vaccine. The pigs were monitored daily for temperature, body weight, fecal consistency scores, physical condition, average daily weight gain and mortality. Animals were also monitored for shedding of the vaccine and challenge organisms. All animals were necrospied at termination of the trial and tissues were cultured for the challenge organism.

TABLE 2
Bacterial strains, description, and doses.

| | Relevant | | Dose |
|---|---|---|---|
| Strain | Genotype | Description | (CFU/animal) |
| D1 | Tn::waaK | LPS mutant | 9.8 × 10⁸ |
| H5 | Tn:waa | LPS mutant | 9.7 × 10⁸ |
| χ3781 | Δcya Δ(crp-cdt) | xx mutant | 8.1 × 10⁸ |
| | vpl+ | | |
| P93- | Wild-type | Wild-type challenge | 8.0 × 10⁹ |
| 558 | | strain | |

The pigs were vaccinated orally via the drinking water. Bacterial cultures were diluted in sterile distilled water to a final concentration of 1×10⁹ CFU/ml. Animals were offered 100 ml of the vaccine preparation via waterers for an hour and the amount of water consumed during this period was measured and the actual dose level determined. The pigs were monitored daily for clinical symptoms (% mortality, % morbidity, % diarrhea days, % shedding days, and average daily gain). The response of pigs to the vaccine is summarized in Table 3. No adverse reactions or clinical signs of disease were observed in these animals regardless of the vaccine given. The animals tolerated the vaccine, continued to feed well, and gained weight. The only observable clinical signs observed were vaccinate were a short-term elevation in rectal temperature (at 24 to 72 hours post vaccination) for animals vaccinated with χ3781, and a transient loose stool (1-2 days) for animals vaccinated with H5.

Ante mortem isolates of salmonellae collected after vaccination were typed for identity and confirmed to be serogroup C1. As noted in Table 5, recovery of vaccine serogroup was correlated with the vaccine administered. No salmonellae were recovered from naive animals or those vaccinated with χ3781 during the post-challenge period. Only one of eight animals (12.5%) shed strain D1 during this period, and this was on a single day (at 6 days post-vaccination). Most (75.0%) of the animals vaccinated with strain H5 presented with transient shedding following vaccination. In these animals, shedding began two days after vaccination and occurred for one (4 animals), two (1 animal) or thirteen (1 animal) of the 16 sample days. The overall duration of shedding days in these animals 14.8%, of which most was accounted for by only one of the animals.

The pigs were then challenged with a highly virulent wild type S. choleraesuis (P93-558), which was a field isolate obtained from a case of saimonellosis. Following a 24 hour fast, at 28 days post-vaccination, oral challenge-exposure of the animals was via the feed by mixing 10 ml of the bacterial cultures into 200 grams of gruel mixture composed of approximately 50% feed and 50% non-chlorinated water for a final challenge dose of 8×10⁹ virulent S. Choleraesuis.

The response of animals to such challenge exposure is summarized in Table 3. All animals given the placebo presented with pyrexia that was accompanied by a severe watery diarrhea. They became anorexic, listless and dehydrated and 50% died within three to eighteen days of challenge. These animals shed the challenge strain for most of the post-challenge period (80.4% shedding days). In contrast, vaccinates were more resistant to infection than naive animals (Table 3). There was a significant reduction in both the severity and duration of morbidity, mortality, days of inactivity, diarrhea, and shedding of the challenge organism depending on the vaccine given. Overall, animals vaccinated with the waa mutants (strains D1 and H5) were the most refractory to challenge exposure, had the most weight gain, and the lowest number of shedding days (Table 5). A reduction in both the numbers of shedding days and clinical scores following challenge exposure was also noted with strain χ3781 was also observed. However, the clinical scores and weight gain for these animals were between those of the naïve-challenged animals and those vaccinated with the waa mutants (Table 4). This vaccine did not lower the temperature spike (Table 4), and more overall shedding (50% shedding days) was observed in response to challenge (Table 5) in animals vaccinated with χ3781.

TABLE 3
Clinical Scores during the pre-and post-vaccination periods.

| | | | % | % | % | | |
|---|---|---|---|---|---|---|---|
| | | | Mor- | Mor- | Diarrhea | Shift in | Ave. Daily |
| Vaccine | Time | N = | tality | bidity | Days | Temp. | Gain |
| None | pre | 8 | 0 | 0 | 0 | 39.0 | |
| | post | | 0 | 0 | 1.9 | 39.3 | 0.38 ± 0.07 |
| WaaK | pre | 8 | 0 | 0 | 0 | 39.0 | |
| D1 | post | | 0 | 0.2 | 1.5 | 39.4 | 0.33 ± 0.06 |
| Waa | pre | 8 | 0 | 0 | 0 | 38.8 | |
| H5 | post | | 0 | 1.5 | 6.7 | 39.2 | 0.32 ± 0.12 |
| χ3781 | pre | 8 | 0 | 0 | 0 | 39.1 | |
| | post | | 0 | 0 | 1.8 | 40.7 | 0.34 ± 0.06 |

Pre = the three days prior to challenge (used to determine baseline scores). Post = the 28 day vaccination period.

TABLE 4
Clinical Scores during the pre- and post-challenge periods.

| | | | % | % | % | | |
|---|---|---|---|---|---|---|---|
| | | | Mor- | Mor- | Diarrhea | Shift in | Ave. Daily |
| Vaccine | Time | N = | tality | bidity | Days | Temp. | Gain |
| None | pre | 8 | 0 | 0 | 2.9 | 39.0 | |
| | post | | 50 | 34.6 | 62.7 | 40.3 | 0.05 ± 0.5 |
| D1 | pre | 8 | 0 | 0 | 4.2 | 39.2 | |
| | post | | 0 | 10.1 | 19.0 | 39.9 | 0.69 ± 0.10 |
| H5 | pre | 8 | 0 | 3.6 | 7.1 | 39 | |
| | post | | 0 | 8.3 | 20.8 | 39.9 | 0.67 ± 0.14 |
| χ3781 | pre | 8 | 0 | 0 | 4.8 | 39.2 | |
| | post | | 0 | 23.2 | 37.5 | 40.3 | 0.56 ± 0.19 |

Pre = the three days prior to challenge (used to determine baseline scores). Post = the 28 day vaccination period.

TABLE 5
Fecal excretion of vaccine and challenge organisms.

| | % Shedding Days | % Shedding Days |
|---|---|---|
| Vaccine | (of the vaccine) | (of the challenge organism) |
| None | 0 | 80.4 |
| D1 | 0.8 | 34.6 |
| H5 | 14.8 | 30.9 |
| χ3781 | 0 | 50.0 |

B. Bacteriologic Examination at Necropsy

The frequency of recovering the challenge organism from intestinal tissues and contents, mesenteric lymph nodes, and internal organs at necropsy are shown in Table 6. From naïve animals, the challenge organism was recovered from 7 of the 8 animals (87.5%) tested. In addition, analysis of the number of organs that were culture positive at necropsy was 4.5 overall. In contrast, although all vaccinates had nearly as many animals colonized (75, 62.5 and 75% of animals vaccinated with strain D1, H5, or χ3781, respectively) with the challenge organism, the overall tissue burden was 2.8 to 3.6 times lower than naïve animals (Table 6).

TABLE 6
Recovery % of S. enterica serovar Choleraesuis
challenge organism in tissues at necropsy.

| | | | | | | | Mean | Rec- |
|---|---|---|---|---|---|---|---|---|
| | | Liv- | | | | | No. | tal |
| Vaccine | Lung | er | Spleen | MLN | ICV | Cecum | Tissues | swab |
| None | 25 | 50 | 25 | 50 | 87.5 | 75 | 4.5 | 50 |
| D1 | 0 | 0 | 12.5 | 0 | 12.5 | 12.5 | 1.25 | 12.5 |
| H5 | 0 | 0 | 0 | 12.5 | 25 | 12.5 | 1.375 | 12.5 |
| χ3781 | 0 | 0 | 0 | 12.5 | 25 | 37.5 | 1.625 | 12.5 |

MLN = mesenteric lymph nodes; ICV = ileocecal valve; Col Con = colonic contents

D. Efficacy of a S. Typhimuriunm waaK Mutant as a Vaccine in Cattle
(2051-7923-I-MJK-98-006)

The safety and efficacy of a live-attenuated S. Typhitnurium waaK mutant as a vaccine was determined in cattle (6 calves per group, 10-14 days of age at vaccination). After incubation for 18-24 hr at 37° C., colonies from a heavy growth area were swept with a sterile loop and inoculated into LB broth. After 14 hrs of static incubation at 37° C., 1.0 ml of this culture was used to innoculate 22.5 ml of fresh LB broth in 250 ml sterile polycarbonate Erlemeyer flasks. After 6 hrs of static incubation at 37° C., 2.5 ml of the resulting undiluted broth culture was added to 3.0 liters of milk replacer for administration to each calf. Dilution and viable plate count on blood agar determined “Numbers of Viable Bacteria” for each strain at the time of preparation. Each vaccine was maintained at room temperature and delivered to animals as soon as possible after preparation (within ˜30 minutes). Baseline temperatures and clinical scores (mortality, physical condition, inactivity, diarrhea (fecal score), and shedding of bacteria) were recorded on Days 1-4. The calves were vaccinated orally via the milk replacer on Day 4 with either wild type or a mutant bacteria at a dose of ˜1×10⁹ CFUs/calf. For oral vaccination, 1 ml of the lab grown vaccine culture was innoculated in the calf s milk replacer. The number of CFUs per ml was determined by performing serial 10-fold dilutions of the final formulation, and plating on agar. The dose per animal was then determined by multiplying the number of CFUs/ml by the number of mls consumed by the animal, giving a final vaccine dose of ˜1×10⁹ CFUs/calf. Because each calf consumed its entire amount of milk replacer on the day of vaccination, the number of CFUs per animal was the same as the number of CFUs/ml of culture.

The calves were monitored daily for clinical symptoms (% mortality, physical condition, % inactive days, fecal score, and 5 shedding days) for 28 days post-vaccination (Days 5-32), of which Days 29-32 were considered a baseline before challenge with wild type bacteria. If a calf died during the period of interest, it was assigned a score of “1” for the mortality variable, otherwise, the mortality variable assigned was “0”. The physical condition was scored on a scale of 1 to 5, where “1” was a healthy, active animal with normal hair-coat; “2” was a mildly depressed animal that was intermediate in activity and had a rough hair-coat; “3” was a moderately to severely depressed animal that was inactive/lethargic and/or gaunt irrespective of hair-coat; “4” was a moribund animal; and “5” was a dead animal. If a calf died, the physical condition was assigned a “5” for the day of death (or the following day depending on the time of death), and missing values thereafter. The average physical condition was taken as the average of the daily scores within the period of interest for each calf. The average physical scores were then used to calculate the rescaled score in the following way: the resealing score=100×(average physical condition score−1)/4. This converts the 1-5 scale into a 0-100 scale. The % inactive days score was determined by calculating the percent of days during the period of interest that a calf had a score of greater than 2 on the physical condition score. The fecal score was scored on a scale of 1-4 where “1” is normal, solid formed or soft with form; “2” is soft unformed; “3” is watery with solid material; and “4” is profuse watery/projectile with little or no solid material. The % shedding days was calculated as the percent of days during the period of interest that a calf had a rectal swab positive for Salmonella.

The calves were then challenged with a highly virulent, heterologous wild type S. thyphimurium (B94-019) at 28 days post-vaccination (Day 32). The calves continued to be monitored for clinical symptoms for a further 14 days post-challenge (Days 33-46). Results post-vaccination (and pre-challenge) are displayed in Table 7 below. Results post-challenge are displayed in Table 8 below. Necropsy was performed on Day 46 or at death, and tissue and fecal samples were obtained for culture of the challenge organism.

Animals vaccinated with the waaK mutant became inactive, lost weight, developed pyrexia, had profuse diarrhea with in 2 to 7 days post infection. Two animals from this group died during this period. Calves given a low dose of the wild-type parent strain developed diarrhea and were slightly depressed but did not show other clinical signs. The mean maximum increase in rectal temperature was 1.74 and 1.59 for animals given the waaK mutant and wild type strain, respectively.

TABLE 7
Response of calves to vaccination with waaK mutant
S. typhimurium vaccines.

| Vac- | | | Mor- | | % | | % |
|---|---|---|---|---|---|---|---|
| cine/ | | | tality | Physical | Inactive | Fecal | Shedding |
| Strain | Time | N = | (%) | Condition | Days | Score | Days |
| None | pre | 6 | 0 | 0.0 | 0 | 1.0 | 0.0 |
| | post | | 0 | 0.4 | 0 | 1.4 | 7.6 |
| | (1-28) | | | | | | |
| WaaK | pre | 6 | 0 | 0.0 | 0 | 1.2 | 0.0 |
| | post | | 33.3 | 1.5/11.5 | 14.2 | 1.6 | 72.4 |
| | (1-28) | | | | | | |
| ΔssaJ | pre | 6 | 0 | 0.0 | 0 | 1.3 | 0.0 |
| | post | | 0 | 0.0 | 0 | 1.4 | 57.6 |
| | (1-28) | | | | | | |
| wild- | pre | 6 | 0 | 1.0 | 0 | 1.0 | 0.0 |
| type | post | | 0 | 0.0 | 0 | 1.6 | 58.8 |
| | (1-28) | | | | | | |

E. Efficacy of a S. typhimurium waaK Mutant as Vaccines in Cattle
(2051-7923-I-MJK-98-006)

Twenty-eight days after vaccination with the S. thyphimurium waaK mutants, calves were challenged with a high dose (1.3×10⁹) of a virulent strain of S. thyphimurium. The response of calves to this extreme challenge exposure is shown in Table 8. All naïve animals exhibited pyrexia, which was accompanied by a severe watery diarrhea, listlessness, anorexia and dehydration. All non-vaccinated animals excreted the challenge organism for 100% of their live days post-challenge.

In contrast animals vaccinated with waaK mutant had fewer days of inactivity, duration of diarrhea, lower temperature responses, and showed a reduction in shedding of the challenge organism.

TABLE 8
Reduction in clinical signs in vaccinates post-challenge
showing the efficacy of S. typhimurium vaccines.

| | | | | | | % |
|---|---|---|---|---|---|---|
| Vaccine/ | | Mortality | Physical | % Inactive | Fecal | Shedding |
| Strain | N = | (%) | Condition | Days | Score | Days |
| None | 6 | 100.0 | 50.5 | 73.9 | 3.0 | 100 |
| waaK | 6 | 75.0 | 43.2 | 41.7 | 2.7 | 94.6 |
| ΔssaJ | 6 | 83.3 | 38.6 | 43.1 | 2.8 | 94.0 |
| wild-type | 6 | 66.7 | 51.8 | 61.0 | 3.1 | 91.7 |

The data from culturing of tissue (>2g) or fecal (>2g) samples showed that there was a reduction of the challenge strain in the tissues from animals vaccinated with the waaK mutants compared to the naive controls (Table 9), and that oral administration of each of these three mutants as a vaccine was safe and efficacious against experimentally induced saimonellosis.

TABLE 9
Recovery (%) of various S. Typhimurium isolates
in tissues at necropsy

| Vaccine | N | Cecum | Feces | MLN | Lung | Liver | Spleen |
|---|---|---|---|---|---|---|---|
| None | 6 | 100.0 | 83.3 | 100.0 | 100.0 | 100.0 | 100.0 |
| waaK | 6 | 50.0 | 50.0 | 50.0 | 66.7 | 50.0 | 50.0 |
| ssaJ | 6 | 100 | 83.3 | 83.3 | 83.3 | 83.3 | 83.3 |
| wild type | 6 | 100 | 100.0 | 100.0 | 83.3 | 83.3 | 83.3 |

Numerous modifications and variations of the above-described invention are expected to occur to those of skill in the art. Accordingly, only such limitations as appear in the appended claims should be placed thereon.

1-16. (canceled)

17. A vaccine composition comprising an immunologically protective amount of a first attenuated mutant Salmonella bacterium comprising an inactivated waaK gene.

18. The vaccine composition of claim 17 wherein said waaK gene is inactivated by disruption of a portion of the gene.

19. The vaccine composition of claim 18 wherein said disruption occurs by an insertion mutation.

20. The vaccine composition of claim 18 wherein said disruption occurs by a deletion mutation.

21. The vaccine composition of claim 18 wherein said disruption occurs by a substitution mutation.

22. The vaccine composition of claim 17 wherein the inactivated gene is selected from the group consisting of:
(a) the waaK gene set forth in SEQ ID NO: 1;
(b) a full length nucleotide sequence that hybridizes to the non-coding complement of SEQ ID NO: 1; and
(c) a full length Salmonella nucleotide sequence that has 80% sequence identity to SEQ ID NO: 1.

23. The vaccine composition of claim 17 wherein said bacterium comprises an inactivated waaK gene and a second inactivated virulence gene.

24. The vaccine composition of claim 22 further comprising a second attenuated mutant Salmonella bacterium in which one or more virulence genes have been inactivated.

25. The vaccine composition of claim 24 wherein said first and second mutant Salmonella bacteria are from different serogroups.

26. The vaccine composition of claim 24 wherein said Salmonella bacteria are from any of serogroups A, B, C, D, or E.

27. The vaccine composition of claim 26 wherein said serogroup is selected from any of serogroups A, B, C₁, C₂, D₁, and E₁.

28. The vaccine composition of claim 17 wherein said attenuated mutant Salmonella bacterium further comprises a polynucleotide encoding a non-Salmonella polypeptide.

29. A method of conferring protective immunity on an animal comprising the step of administering to said animal a vaccine composition comprising an immunologically protective amount of an attenuated mutant Salmonella bacterium comprising an inactivated waaK gene.

30. The method of claim 29 wherein said attenuated mutant Salmonella bacterium is non-reverting.

31. The method of claim 29 wherein said immunologically protective amount of said attenuated bacterium provides an improvement in mortality, symptomatic diarrhea, physical condition, or milk production.

32. The method of claim 29 wherein said waaK gene is inactivated by a disruption of a portion of the gene.

33. The method of claim 29 wherein said disruption occurs by an insertion mutation.

34. The method of claim 29 wherein said disruption occurs by a deletion mutation.

35. The method of claim 29 wherein said disruption occurs by a substitution mutation.

36. The method of claim 29 wherein said animal is selected from the group consisting of cattle, sheep, goats, horses, pigs, poultry and other birds, cats, dogs, and humans.

37. The method of claim 29 wherein said animal is a mammal.

38. The method of claim 29 wherein said animal is a pig.

39. The method of claim 29 wherein said animal is a cow.

40. The method of claim 29 wherein said animal is a bird.

41. The method of claim 29 wherein said animal is a horse.

42. A method of delivering a polypeptide antigen to an animal comprising the step of administering the vaccine composition of claim 22 to said animal.

43. The vaccine composition of claim 22 wherein said Salmonella bacteria are Salmonella typhimurium.

44. The vaccine composition of claim 22 wherein said Salmonella bacteria are Salmonella choleraesuis.

45. The vaccine composition of claim 22 wherein said Salmonella bacteria are Salmonella dublin.

46. The vaccine composition of claim 17 wherein said attenuated mutant Salmonella bacterium is non-reverting.

47. The method of claim 37 wherein said mammal is a human.


SEQUENCE LISTING (5 sequences)

SEQ ID NO: 1 - DNA, 1160 bp, S. typhimurium
tcacttatcaaaccagtttttcatttgttcctcgaaacgctgcgctacattttcccaactgtattttgaaaacaccagggattttgctttttcggcaatctggtggcgttccttatcagcaagcgcacggttaatatcattaattatactgtcgctcgacataggttctgcgaggtgatagcccgttatgccatctaacacaaattcgctaatccccccttttttgctggcaagaaccgcttttcctgctgccatcgcttctacagccaccatgcaaaatgcttcttcaacctgagatggcacaataaccagatcggctatatgatagaagttatgcatctggtcaggagattgccccccagccataatacaatccgttccaatctcttttgcggcgtccagtactttcttttgatactctgctttttcacccttgcggcttgcataagggtcgccaacaacgacaagtttaatattacttcttaaggtacgtaattgtttgaacgcctgcaaaagcaacaggatgcctttatcaggcgaaattctcccggcatacaagagaacggtggcatcttccgcaatatttaattgctgacgaagattatcttgtgggtttcttttataagtctcagcacaaaaaccattaggcacaatactaacagcagcggcgggcaatctttcttcataaaacgctttaagaaactgactgggcacgataatttttgcatcattatcaggaagttctggttcaaatgcattatgcatgtgcataaccagttttgcattcggattgcgctctctgatctgccgatacagtttcatactattatgaataacaatgacgctatcttcctgggtagtcactttatctctaatattaaggatgcgctgggaatagggtagtgggtcgagacgagtccatttctgaaaaagacgcttataaactttactaaacccgatgtaatgaatatcacagttatcgtttattttattatattcaggatagccagcattctttatacaagcaatagcattcggtattgatagtcgttttgcaacctggtaaatccaggtttctaccgcagccgcaccacgaggaggaattgaaaatataggagtaacagtaaatatgatttttttaatcataatagctataatcc

SEQ ID NO: 2 - DNA, 35 bp, Artificial sequence
  Primer
gccaagtcgacatagtaggtgttctgtgggcaata

SEQ ID NO: 3 - DNA, 48 bp, Artificial sequence
  Primer
ttctggattatagctattatgattgtttgataagtgattgagtcctga

SEQ ID NO: 4 - DNA, 48 bp, Artificial sequence
  Primer
tcaggactcaatcacttatcaaacaatcataatagctataatccagaa

SEQ ID NO: 5 - DNA, 35 bp, Artificial sequence
  Primer
gccaagtcgacgtgtacgaacaggcttcagtggat